(19) United States
(12) Patent Application Publication
(10) Pub. No.: US 2011/0082485 A1
(43) Pub. Date: Apr. 7, 2011

(54) CUTTING BLADE FOR MORCELLATOR

(75) Inventors: Martin J. Nohilly, Murray Hill, NJ (US); Anthony S. Miksza, Nazareth, PA (US); Robert Nering, Stockton, NJ (US)

(73) Assignee: Ethicon, Inc., Somerville, NJ (US)

(21) Appl. No.: 12/954,280

(22) Filed: Nov. 24, 2010

Related U.S. Application Data
(63) Continuation of application No. 11/502,340, filed on Aug. 10, 2006, now abandoned.

(51) Int. Cl. A61B 17/32

(52) U.S. Cl. 606/167

(57) ABSTRACT

A cutting blade for a surgical morcellator has a distal end and a sharpened cutting edge situated at the distal end for transecting tissue to form tissue morsels, the cutting blade being structured to minimize contact between the sharpened edge and a tissue grasping instrument used with the morcellator during a surgical procedure. The cutting blade includes a generally cylindrically-shaped sidewall defining an axial bore for the passage of transected tissue morsels therethrough, the sidewall having an outer surface residing generally in a first cylindrical plane, an inner surface residing generally in a second cylindrical plane, and a sloped surface, the inner surface of the sidewall being disposed radially inwardly of the outer surface of the sidewall, the sloped surface extending transversely between the inner surface and the outer surface in the direction of the distal end of the cutting blade and joining the outer surface at an acute angle to define with the outer surface the sharpened cutting edge, the sharpened cutting edge residing in the first cylindrical plane in which the outer surface of the sidewall generally resides, wherein the sloped surface of the cutting blade sidewall extends radially inwardly beyond the second cylindrical plane in which the inner surface of the cutting blade sidewall generally resides to define a circular protrusion situated radially inwardly of the inner surface for selective engagement with the tissue grasping instrument used with the morcellator during a surgical procedure.

### CROSS REFERENCE TO RELATED APPLICATIONS

The present application is a continuation of U.S. patent application Ser. No. 11/502,340, filed Aug. 10, 2006. The disclosure of the above-identified patent application is hereby incorporated by reference herein.

### BACKGROUND OF THE INVENTION

1. Field of the Invention

The present invention relates generally to surgical devices and methods, and more particularly to a laparoscopic morcellator having a detachable handle and various other improved features.

2. Description of the Related Art

Minimally invasive surgical procedures, such as laparoscopic procedures, have become very common. These procedures typically involve one or more small incisions that provide access to the relevant internal organ or tissue. A trocar, cannula or the like is placed into each incision, and all surgical steps are subsequently performed with instruments passed through or into the trocar(s).

Many times it is desirable to remove relatively large masses of tissue, for example a uterine fibroid, which can be difficult and time consuming given the diameter of the trocar. To this end, laparoscopic morcellators have been developed to assist in severing the tissue mass into pieces that can readily be removed through the trocar. An example of one such a morcellator is described in detail in U.S. Pat. No. 6,039,748, which is incorporated herein by reference in its entirety.

Known morcellators typically include a rotating tube having a sharp distal cutting edge, which rotates within an outer stationary tube. The morcellator is inserted through a cannula or trocar, or more commonly directly through the incision. A grasping instrument (i.e., tenaculum) is inserted through the inner rotating tube. Using the tenaculum, the surgeon pulls the tissue to be severed up into the tube so that the rotating edge of the inner tube severs the grasped portion of tissue. By repeating the grasping and severing procedure, the surgeon can remove the large tissue mass in increments.

Another technique surgeons have developed to improve the speed of tissue removal using a morcellator is known as “orange peeling.” In orange peeling, the cylindrical blade of the morcellator is held on a plane with the outside of the organ or tissue being removed in such a way as to allow the organ or tissue to be rotated. This allows a longer strip to be removed as opposed to the “coring” technique described above, which limits the length of the strip removed to the thickness of the organ. Orange peeling requires skill of the surgeon holding the morcellator as well as skill of the assistant that is passing tissue to the morcellator with a second grasper in the cavity. The skill required is in keeping the blade at the surface of the tissue without either allowing the blade to dive in, or “core”, and at the same time not leaving the surface so much that the tissue strip becomes thin or breaks. Orange peeling is better from a safety standpoint as well, as the blade remains visible at all times to the user. Thus, it would be desirable to provide a morcellator having improved feature(s) that facilitate the ability of the surgeon to use the orange peeling technique.

Another difficulty sometimes encountered with known morcellators is that during use, whether by coring or orange peeling, the amount of tissue being withdrawn can cause friction within the inner rotating tube or to the seal system during removal. The larger the tissue sections or strips, the more exaggerated this problem becomes. It would further be desirable to provide a morcellator that lowers such withdrawal forces.

In addition to friction encountered during tissue removal, manipulation of the grasping instrument within the rotating inner tube can interfere with the blade rotation and tends to lead to dulling of the blade with known morcellators, since the sharp edge is positioned on the inner most point on the circumference of the inner tube. It would also be desirable to provide a morcellator that provides increased protection against such interference and blade dulling.

Finally, as indicated above, morcellators are typically inserted through a cannula, or more commonly directly through the incision. When inserted directly into the incision the existing trocar must first be removed. Following morcellation, if any other procedures or tasks are to be performed within the cavity, the morcellator must be removed before any other laparoscopic instrument can be inserted through that same portal. Removal and reinsertion of trocars and laparoscopic instruments during a given procedure is awkward and time consuming, and creates additional trauma at the site. It is further desirable to provide a morcellator that will greatly reduce the need for such exchanges.

### SUMMARY OF THE INVENTION

It is an object of the present invention to provide a morcellator cutting blade which minimizes the chances of the blade becoming dull or damaged during use.

It is another object of the present invention to provide a cutting blade for a morcellator which minimizes the chance of transected tissue morsels from becoming dislodged within the morcellator.

It is yet a further object of the present invention to provide a cutting blade for a morcellator which may be at least partially formed from less expensive materials than those used in conventional morcellators.

It is still a further object of the present invention to provide a cutting blade for a morcellator which overcomes the inherent disadvantages of conventional morcellator cutting blades.

In accordance with one form of the present invention, a cutting blade for a morcellator is generally cylindrical in shape and includes a sidewall having an inner surface and an outer surface and which defines an axial bore radially inwardly of the inner surface thereof. The inner surface and the outer surface of the cutting blade at least partially reside in concentric cylindrical planes. The inner surface includes a sloped or beveled portion situated at a distal end of the cutting blade, which slope portion extends toward the outer surface in the direction of the distal end of the cutting blade to define a sharpened edge at the intersection of the inner and outer surfaces. The sharpened edge preferably resides in the cylindrical plane of the outer surface of the cutting blade. The sloped or beveled edge of the inner surface is provided to engage the tenaculum if it is not obvious to the surgeon that the tenaculum claws are spread to such an extent that they will contact the cutting blade. Under such circumstances, the tenaculum will contact the sloped or beveled edge of the inner surface of the cutting blade, rather than the sharpened edge, and therefore not dull or damage the sharpened edge of the cutting blade.

In another form of the present invention, the cutting blade is formed of two sections. The first section, the distal end of the cutting blade, is formed with a sharpened edge having a first diameter, and the second section which is situated adjacent to the distal end portion of the cutting blade has an inner surface having a second diameter. The second diameter of the second section is greater than the first diameter of the sharpened edge of the distal end portion of the cutting blade so that transected tissue morsels cut by the rotating distal end portion of the cutting blade should not become dislodged from the tenaculum or cause undue friction with the inner surface of the cutting blade as the tenaculum pulls the tissue morsel through the axial bore of the rotating cutting blade of the morcellator.

These and other objects, features and advantages of the present invention will be apparent from the following detailed description of illustrative embodiments thereof, which is to be read in connection with the accompanying drawings.

These and other preferred embodiments of the present invention will be described in more detail below.

### BRIEF DESCRIPTION OF THE DRAWING

FIG. 1 is cross-sectional view of the distal end portion of a conventional morcellator cutting blade and the end portion of a tissue grasping instrument used with the morcellator during a surgical procedure.

FIG. 2 is a cross-sectional view of the distal end portion of a morcellator cutting blade formed in accordance with the present invention, as well as the end portion of a tissue grasping instrument used with a morcellator employing the cutting blade of the present invention during a surgical procedure.

FIG. 3 is a cross-sectional view of the distal end portion of a cutting blade constructed in accordance with a second form of the present invention, as well as the end portion of a tissue grasping instrument used in conjunction with a morcellator employing the cutting blade of the present invention during a surgical procedure.

FIG. 4 is an enlarged cross-sectional view of a portion of the cutting blade of the present invention shown in FIG. 3.

FIG. 5 is a cross-sectional view of the distal end portion of a cutting blade constructed in accordance with a third form of the present invention, as well as the end portion of a tissue grasping instrument used in conjunction with a morcellator employing the cutting blade of the present invention during a surgical procedure.

FIG. 6 is an enlarged cross-sectional view of a portion of the cutting blade of the present invention shown in FIG. 5.

FIG. 7 is a cross-sectional view of the distal end portion of a cutting blade constructed in accordance with a fourth form of the present invention, as well as the end portion of a tissue grasping instrument used in conjunction with a morcellator employing the cutting blade of the present invention during a surgical procedure.

FIG. 8 is an enlarged cross-sectional view of a portion of the cutting blade of the present invention shown in FIG. 7.

FIG. 9 is a perspective view of the distal end portion of a cutting blade for a morcellator constructed in accordance with a fifth form of the present invention.

FIG. 10 is a side view of the distal end portion of the cutting blade of the present invention shown in FIG. 9.

FIG. 11 is a side view of the distal end portion of a cutting blade for a morcellator constructed in accordance with a sixth form of the present invention.

FIG. 12 is a cross-sectional view of the distal end portion of a cutting blade for a morcellator constructed in accordance with a seventh form of the present invention.

FIG. 13 is an enlarged cross-sectional view of a portion of the cutting blade of the present invention shown in FIG. 12.

### DETAILED DESCRIPTION

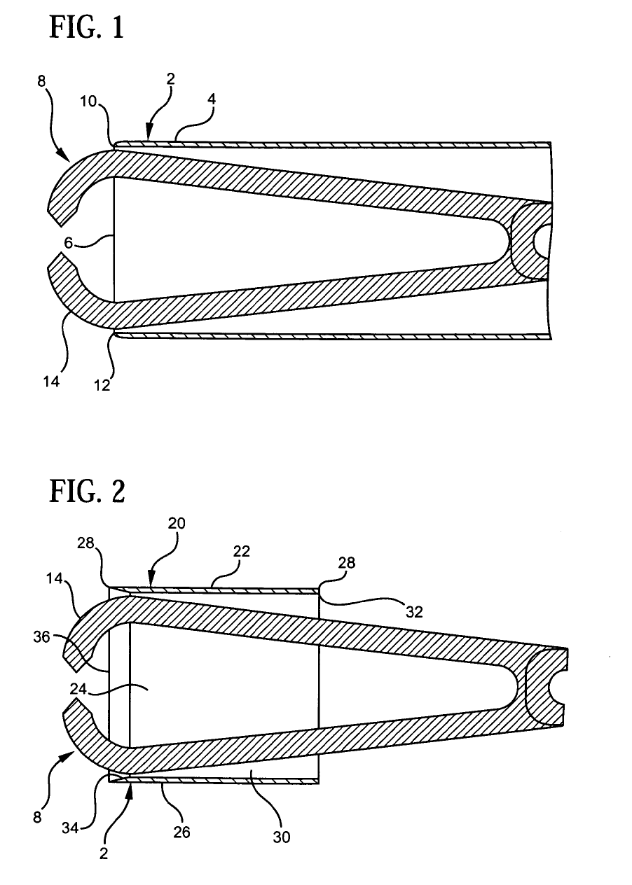

To facilitate an understanding of the present invention, reference should be made to FIG. 1 of the drawings, which shows the distal end 2 of a cutting blade 4 of a conventional morcellator. The morcellator has a cylindrical cutting blade 4 that rotates within an outer fixed tube or sleeve (not shown). The cutting blade 4 defines an axial bore 6 in which is selectively received a tissue grasping tool, or tenaculum 8. The exposed cutting edge 10 of the conventional morcellator blade 4 is sharpened in the cylindrical plane of the radially inner surface 12 of the cutting blade, as can be seen in FIG. 1.

The tenaculum 8 includes two expandable grasping claws or hooks 14 which are intended to grasp the tissue of an anatomical body (e.g., organ) and pull the tissue toward the rotating cutting blade 4 of the morcellator so that it may be cut into tissue “morsels”. The claws 14, after grasping tissue between them, may be in an expanded or spread state, as the tenaculum 8 is pulled toward and through the axial bore 6 of the cutting blade in order to transect the tissue and remove the severed tissue morsel from the patient's body through the axial bore 6 of the cutting blade. When the tenaculum claws 14 are in this expanded state, it is possible for them to contact the sharpened cutting blade edge 10 of the morcellator. Both the tenaculum 8 and the cutting blade 4 of the morcellator are made from metal, preferably stainless steel, and this metal-to-metal contact dulls the blade during insertion and withdrawal of the tenaculum respectively into and from the morcellator. A damaged or dull blade can excessively prolong the laparoscopic surgery using the morcellator. Alternatively, the tenaculum 8 may be off center with respect to the axis of the morcellator cutting blade 4 and contacts the sharpened cutting edge 10 when the tenaculum grasps tissue and pulls it toward the morcellator.

In accordance with one form of the present invention, and as shown in FIG. 2 of the drawings, a cutting blade 20 for a morcellator is formed as an elongated tubular member having a cylindrically-shaped sidewall 22 defining an axial bore 24 for the passage of transected tissue morsels therethrough. The sidewall 22 has an outer surface 26 residing in a first cylindrical plane 28 and an inner surface 30 disposed radially inwardly of the outer surface 26 and residing in a second cylindrical plane 32 concentrically situated within the first cylindrical plane 28. The sidewall 22 further includes a beveled or sloped surface 34 that extends from the inner surface 30 toward the outer surface 26 in a direction toward the exposed free end of the cutting blade to define a sharpened edge 36 in the first cylindrical plane 28 in which the outer surface 26 resides.

The purpose of having the sharpened edge 36 on the outside surface 26 of the cutting blade, as opposed to the inside surface 12 on conventional morcellator cutting blades, is that, when the tenaculum 8 is withdrawn through the morcellator, with its claws 14 spread to some degree, it will contact the inside surface 30 of the cutting blade 20 and not the sharpened edge 36, as can be clearly seen in FIG. 2 of the drawings.

The slope of the beveled surface 34 is preferably about 15 degrees measured as the acute exterior angle A formed between the sloped surface and the first cylindrical plane 28 in which the outer surface 26 of the morcellator blade sidewall 22 generally resides. This angle is preferred as a compromise between obtaining a sharp edge on the cutting blade 20 and protecting the cutting edge 36 against inadvertent contact with the claws 14 of the tenaculum 8.

More specifically, it may not be obvious to a surgeon when viewing the surgical procedure through an endoscope that the claws 14 of the tenaculum are spread to such an extent that the tenaculum 8 will contact the cutting blade 20 if the tenaculum is withdrawn through the morcellator. The angle of the beveled surface 34 of the cutting blade 20 is chosen to be preferably about 15 degrees to protect the sharpened edge 36 against such inadvertent contact with the tenaculum 8 under such circumstances when it is not readily apparent to the surgeon that contact between the tenaculum and the sharpened edge will occur, and still provide a sharp edge for cutting. When it is clearly obvious to an astute surgeon that the tenaculum 8, when grasping tissue for transection, is open to such a degree that it will contact the rotating sharpened edge 36 of the morcellator blade 20, the surgeon will not attempt to pull the tenaculum through the morcellator—he or she will release the tissue and grasp a smaller quantity. Accordingly, the preferred 15 degree slope is chosen as a precautionary angle to protect the cutting blade 20 when it is not so obvious to the surgeon that contact between the tenaculum 8 and the sharpened blade edge 36 will occur. This angle is based on experimentation and observation and may vary widely.

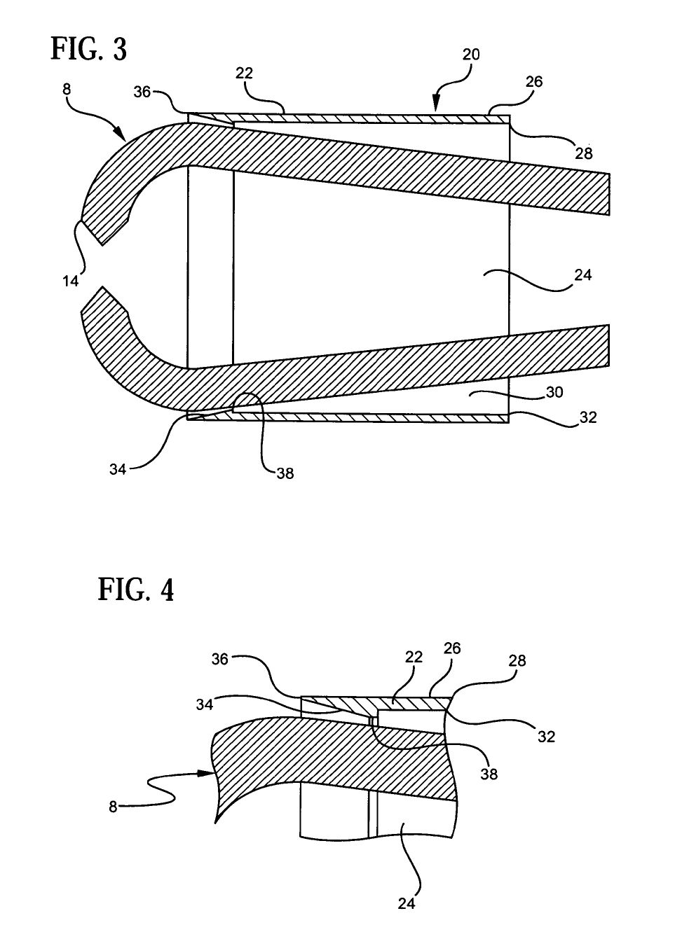

FIGS. 3 and 4 of the drawings illustrate another form of a cutting blade 20 for a morcellator constructed in accordance with the present invention. Here, the beveled or sloped surface 34 of the cutting blade sidewall 22, near the distal free end of the blade 20, continues beyond the general cylindrical plane 32 of the inner surface 30 to define a shoulder 38 that projects radially into the axial bore 24 of the cutting blade. This shoulder 38 can be fabricated integrally with the cutting blade sidewall 22, or separately and mounted thereon, and can be continuous or interrupted circumferentially about the inner surface 30 of the cutting blade sidewall. The shoulder 38, when contacted by the claws 14 of the tenaculum 8, further prevents the tenaculum from engaging the sharpened blade edge 36 which, again, is preferably situated in the cylindrical plane 28 in which the outer surface 26 of the cutting blade sidewall resides, as in the embodiment shown in FIG. 2 of the drawings.

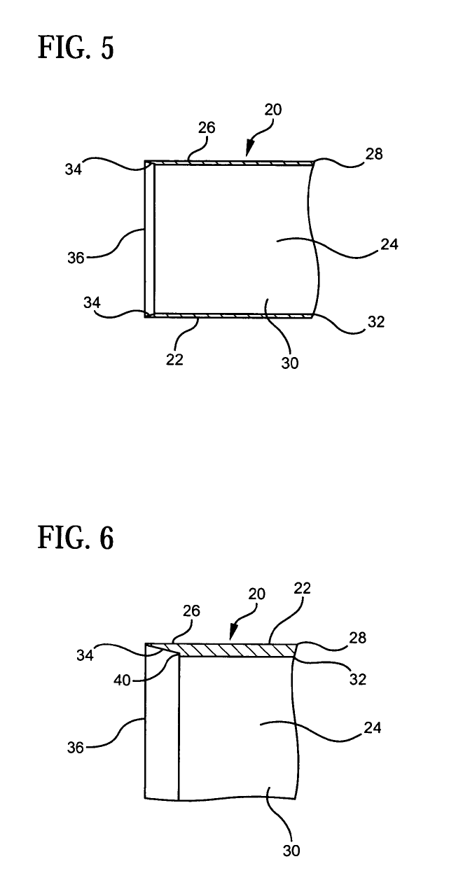

FIGS. 5 and 6 illustrate an alternative form of the cutting blade 20 of the present invention shown in FIGS. 3 and 4. The cutting blade 20 is formed with an edge 40 on the inner surface 30 of its sidewall 22 that radially extends outwardly towards the outer surface 26 of the cutting blade sidewall, where it meets the sloped surface 34. Again, as in the previous embodiments of the present invention, the sloped surface 34 preferably defines with the outer surface 26 a sharpened edge 36 which resides in the cylindrical plane 28 of the outer surface. Thus, the edge 40 defines the cutting blade 20 with a stepped inner surface that is engageable by the claws 14 of the tenaculum 8, rather than the sharpened cutting edge 36 of the blade being contacted, in order to protect the sharpened blade from dulling, becoming outwardly flared or chipping as the tenaculum 8 is withdrawn through or extended from the axial bore 24 of the cutting blade.

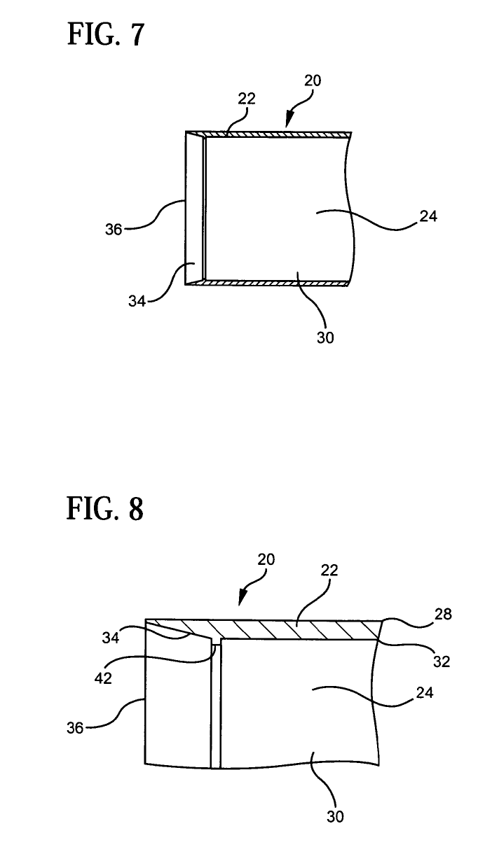

FIGS. 7 and 8 illustrate another form of a morcellator cutting blade 20 constructed in accordance with the present invention. On the inner surface 30 of the cutting blade sidewall 22, in proximity to where the sloped surface 34 meets the inner surface opposite the sharpened cutting edge 36, is situated a raised ring 42 or bump that extends radially into the axial bore 24 of the cutting blade. The ring 42 is again provided so that the claws 14 of the tenaculum 8 will engage the ring 42 rather than contacting the sharpened blade edge 36 and dulling or damaging the cutting edge. The raised ring 42 or bump may be integrally formed with the cutting blade sidewall 22 on the inner surface 30 thereof. Alternatively, the inner surface 30 may include a recess formed therein and circumferentially extending about the cutting blade sidewall 22, into which is captively received and seated therein a portion of the raised ring 42.

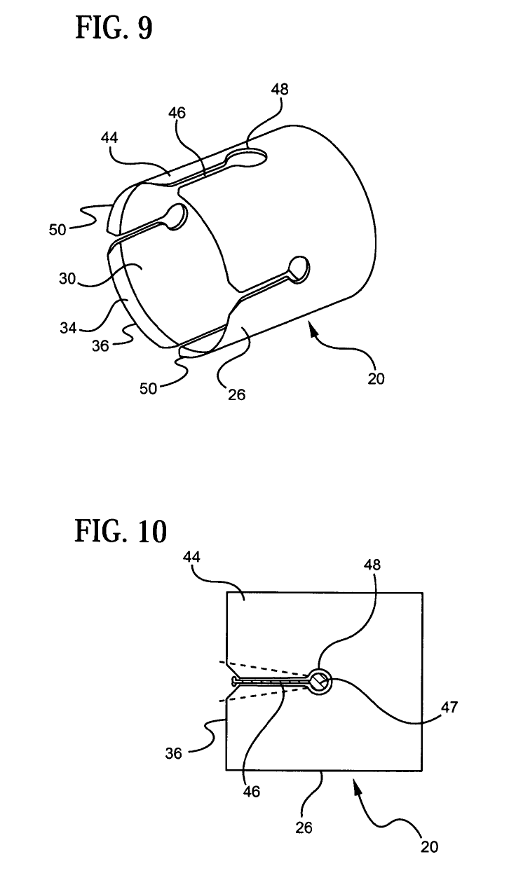

FIGS. 9 and 10 show another form of a cutting blade 20 for a morcellator constructed in accordance with the present invention. In this particular embodiment, at least a portion of the cutting blade is made flexible so that it may deflect when the tenaculum 8 strikes it.

More specifically, and as shown in FIGS. 9 and 10, the distal end portion 44 of the cutting blade 20 is made flexible by forming slots 46 in the blade sidewall 22 extending axially along a portion of the longitudinal length thereof, which slots 46 are spaced apart periodically about the circumference of the cutting blade 20. The slots 46 extend to the sharpened cutting edge 36, and terminate inwardly of the cutting edge in respective round openings 48 formed through the thickness of the cutting blade wall 22. Preferably, as in the other embodiments previously described, a beveled or sloped surface 34 of the cutting blade sidewall 22 extends from the inner surface 30 in the direction of the distal end of the blade and meets the outer surface 26 to define a sharp cutting edge 36 situated in the cylindrical plane 28 in which the outer surface of the cutting blade sidewall resides. However, it is envisioned to be within the scope of the present invention to form the sharpened edge 36 on the inner surface 30 of the cutting blade sidewall 22, with the outer surface 26 joining the sloped surface 34 which extends toward the inner surface to form a sharpened edge 36 on the cutting blade that resides in the cylindrical plane 32 in which the inner surface 30 resides. With this particular embodiment, when the tenaculum 8 and in particular, the claws 14 thereof, engage the cutting blade 20 of the morcellator, the slotted portions 50 of the blade will give or deflect out of the way, so as not to be damaged or dulled when contacted by the tenaculum.

The slots 46 in the cutting blade sidewall 22 may be filled with an elastomeric or polymeric material 47. The slots 46 are filled so that the material 47 is flush with the inner and outer surfaces 30, 26 of the cutting blade sidewall 22. The purpose of filling the slots 46 with an elastomeric or polymeric material 47 is to prevent tissue that is being cut from getting caught in the slots 46 and creating friction or damage to the cutting blade 20, or preventing the cutting blade from rotating within the outer sleeve of the morcellator.

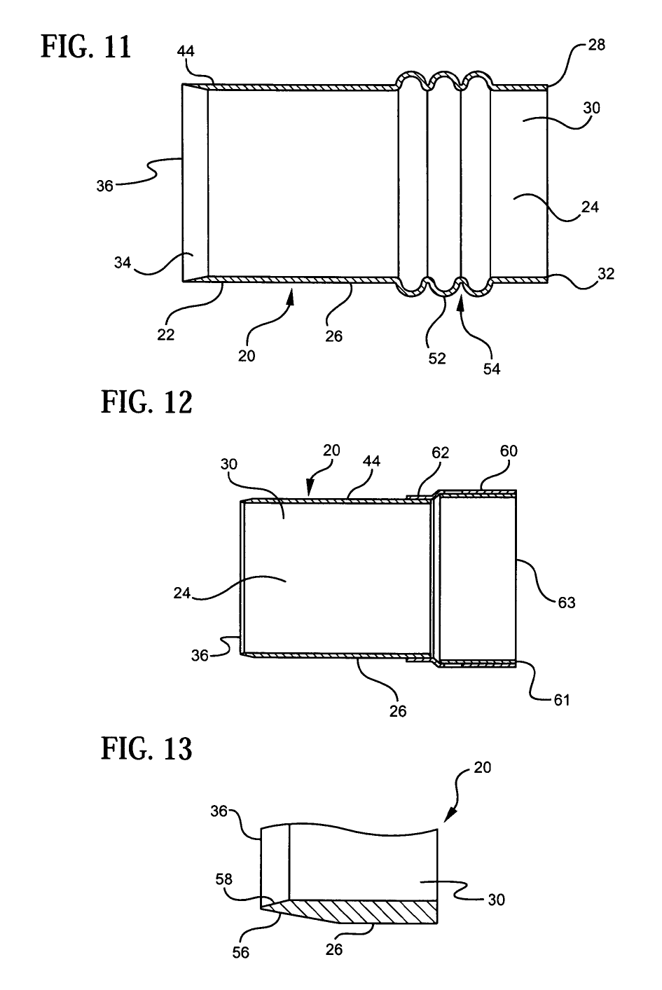

FIG. 11 illustrates yet another embodiment of a morcellator cutting blade 20 formed in accordance with the present invention. Here, the cutting blade 20 is made flexible so that it is self-centering on the tenaculum 8.

More specifically, a portion of the longitudinal length of the cutting blade 20 is formed with undulations 52 in its sidewall 22 to define a flexible joint 54 thereat. The flexible joint 54 allows the distal end of the blade 20 to float and be self-centering on the tenaculum 8. Again, preferably the cutting blade sidewall 22 has a sloped or beveled surface 34 that extends from the inner surface 30 in the distal end direction to the outer surface 26 to form with the outer surface a sharpened cutting edge 36 situated in the cylindrical plane 28 in which the outer surface resides.

The flexible joint portion 54 of the cutting blade may be formed from a separate molded elastomeric or plastic material piece that joins the distal end portion 44 of the cutting blade, which distal end portion 44 may be formed from stainless steel or other metal. Alternatively, the flexible joint 54 may be a series of ripples or undulations 52 formed directly in the metal tubular sidewall 22 of the cutting blade. Oftentimes, the tenaculum 8 is forced to extend at an angle to the axis of the cutting blade 20 to reach anatomical tissue structures. With this embodiment, the cutting blade 20 of the present invention will flex in the direction which the tenaculum 8 extends in order to minimize the chance that the tenaculum will contact the sharpened cutting edge 36 of the blade. The flexible joint 54 a lows the cutting blade to be self-centering on the tenaculum 8 which is situated in the axial bore 24 thereof.

A morcellator cutting blade 20 may also be formed with a sharpened edge 36 disposed between the cylindrical planes 28, 32 in which the inner surface 30 and the outer surface 26 of the cutting blade reside. This particular embodiment is shown in FIGS. 12 and 13 of the drawings.

More particularly, the sidewall 22 of the cutting blade 20 includes a first sloped or beveled surface 56 which extends from the inner surface 30 of the sidewall 22 and is angled toward the outer surface 26 in the direction of the distal end of the cutting blade. Similarly, the sidewall 22 of the cutting blade further includes a second beveled or sloped surface 58 which extends from the outer surface 26 and is angled toward the inner surface 30 in the direction of the distal end of the cutting blade. Together, the first and second sloped surfaces 56, 58 meet to define the sharpened edge 36 of the cutting blade. The sharpened edge 36 is disposed between the cylindrical planes in which the inner surface 30 and the outer surface 26 of the cutting blade sidewall reside. Preferably, the first sloping surface 56 forms an acute exterior angle B with respect to the plane 32 in which the inner surface 30 of the cutting blade sidewall resides, which angle B is about 15 degrees. Similarly, the second sloped surface 58 forms an acute exterior angle C with respect to the plane 28 in which the outer surface 26 resides, which angle C is about 11 degrees. The first sloped surface 56 of the sidewall 22 is provided in this embodiment of the cutting blade, like the previous embodiments described herein, to protect the sharpened edge 36 from the tenaculum 8 when it is not obvious to the surgeon that the claws 14 of the tenaculum are spread apart too wide and would have contacted the sharpened edge 36 of the cutting blade had the cutting blade 20 been formed conventionally with its sharpened edge residing in the cylindrical plane 32 in which the inner surface 30 of the cutting blade sidewall resides.

The distal end of the morcellator cutting blade 20 is preferably made from surgical stainless steel, and even more preferably, a hardened and or coated steel which is easier to keep sharp. Stainless steel is preferred because it will not corrode and provides the axial bore 24 of the cutting blade 20 with a smooth, polished surface which minimizes friction between the cutting blade 20 and transected morsels being pulled through the axial bore 24 of the rotating cutting blade of the morcellator. Even more preferably, the distal end 44 of the cutting blade 20 of the present invention is formed from Nos. 304, 316, 316L, or 420, 465 grade stainless steel, although it is envisioned to be within the scope of the present invention to form the distal end of the cutting blade from other grades of surgical stainless steel and from other materials. Furthermore, the cutting blade 20 may be titanium coated on its inside surface and outside surface for extra durability and/or low friction.

It is also envisioned to be within the scope of the present invention to form the cutting blade 20 of the morcellator from different materials or from different grades of materials. As shown in FIGS. 12 and 13 of the drawings, the distal end portion 44 of the cutting blade may be formed from a rather expensive, surgical stainless steel, and or coated surgical stainless steel to improve the hardness properties, to maintain the sharpness and hardness of the cutting edge 36, while the remainder or adjoining portion 60 of the morcellator blade may be formed from a different material, such as a different grade surgical stainless steel or from a thermoplastic material. For example, the adjoining portion 60 of the cutting blade 20 may be formed from a surgical stainless steel of No. 301, 302, 303, 304 grade or the like, which is not as relatively expensive as the material from which the distal end portion 44 of the cutting blade is formed, as the adjoining portion 60 need not retain its hardness and does not define the sharpened edge. It is preferred, however, that the adjoining portion 60 of the cutting blade still provide a polished inner surface 30 to reduce friction between transected morsels and the rotating cutting blade as the tissue morsels are being pulled through the axial bore 24 of the morcellator cutting blade. The distal end 44 of the cutting blade may be affixed to the adjoining portion 60 by welding or brazing the two materials together to form a unitary joint. Alternatively, and as shown in FIGS. 12 and 13 of the drawings, the mating end of the distal end portion 44 of the cutting blade sidewall 22 opposite the sharpened edge 36 may be at least partially closely received by a sleeve 62 formed by machine rolling the corresponding mating end of the adjoining portion 60 of the cutting blade to a smaller inner diameter that approximates the outer diameter of the distal end portion 44 of the cutting blade sidewall 22. The distal end portion 44 that is closely received by the sleeve 62 of the adjoining portion 60 may be welded or adhesively secured to the sleeve.

As mentioned previously, the adjoining portion 60 of the cutting blade may be also formed from a polymeric or elastomeric material. In this case, the mating end of the stainless steel distal end portion 44 of the blade may be attached to the polymeric or elastomeric adjoining portion 60, for example by being closely received by a sleeve 62 formed on the mating end of the adjoining portion 60 of the cutting blade and adhesively secured thereto in much the same way as described previously. Polymeric and elastomeric materials which may be used to form the adjoining portion 60 of the cutting blade include, but are not limited to, PEEK (polyetheretherketone), Polycarbonate, and Nylon. Furthermore, it would be preferred if the adjoining portion 60 of the cutting blade were formed from a material which is inherently lubricious, or the inner surface 30 thereof were to include a lubricious coating, in order to minimize friction between transected tissue morsels and the inner surface 30 of the cutting blade, as the tissue morsels are being pulled by the tenaculum 8 through the axial bore 24 of the cutting blade.

Another feature of the morcellator cutting blade 20 formed in accordance with the present invention is illustrated by FIGS. 12 and 13 of the drawings. It is known that with conventional morcellator cutting blades 4, friction between transected tissue morsels and the rotating cutting blade may cause the morsels to tear from the tenaculum 8 and become dislodged and entrapped within the axial bore 6 of the cutting blade. This occurs more frequently when long strands of transected tissue resulting from a commonly used surgical technique referred to as “orange peeling” are pulled through the morcellator. The long tissue strands twist and turn within the axial bore 6 of the rotating cutting blade as the tenaculum 8 is being pulled through the morcellator. Also, oversized transected tissue morsels which are too large to move freely within the axial bore 6 of the cutting blade 4 may create excessive friction with the inner surface 12 of the cutting blade, become dislodged from the tenaculum 8 and plug the axial bore 6, which may require the morcellator to be removed from the patient, and the obstructing tissue removed from the morcellator bore. Or, the entrapped tissue morsels must be pushed back out the distal end 2 of the morcellator cutting blade 4 where they can be re-grasped by the tenaculum claws 14 and pulled through the morcellator again. Once entrapped in the axial bore 6 of the morcellator cutting blade 4, the tissue morsel may be difficult to grasp, as the tenaculum claws 14 may not be spread wide enough within the axial bore of the cutting blade to securely engage the entrapped tissue. Also, it may be necessary to remove power to the morcellator in order to prevent the cutting blade 4 from rotating, as the entrapped tissue morsel will spin within the axial bore 6 with the rotating cutting blade, making it even more difficult to extract.

The above-described problem with conventional morcellators has been addressed by the present invention. As shown in FIGS. 12 and 13 of the drawings, the cutting blade 20 of the present invention is preferably formed with a relatively short distal end portion 44 attached to the longer adjoining portion 60 of the cutting blade. The adjoining portion 60 of the cutting blade is formed with an inner diameter that is greater than the diameter of the sharpened edge 36 of the distal end portion 44. For example, the sharpened edge 36 of the distal end portion 44 may have a diameter of about 0.515 inches, with the distal end portion 44 having a relatively short length of about 0.625 inches, whereas the adjoining portion 60 of the cutting blade may have an inner diameter of about 0.553 inches, which is 0.038 inches in diameter greater than that of the sharpened edge 36 of the distal end portion 44 of the cutting blade. The length of the adjoining portion 60 of the cutting blade is made to be much greater than that of the distal end portion 44, and could be from about 5.12 inches (135 mm) to about 6.88 inches (175 mm) in length typically, although it could be shorter or longer than the typical dimensions given, as the adjoining portion 60 must pass through the outer sleeve of the morcellator and the morcellator body. There will be little or no friction between the transected tissue morsel and the sidewall 22 of the distal end portion 44 because of its relatively short length as the tenaculum 8 pulls the morsel through the morcellator, and because the width of the tissue morsel is somewhat determined by the sharpened cutting edge 36 of the smaller diameter distal end portion 44, the tissue morsel should pass freely through the relatively larger diameter axial bore 24 of the longer adjoining portion 60 of the cutting blade. Accordingly, if the adjoining portion 60 of the cutting blade is made with a sufficiently greater inner diameter than the diameter of the distal end portion 44 of the cutting blade measured at the sharpened edge 36 thereof, then transected tissue morsels being pulled through the relatively longer adjoining portion 60 of the cutting blade should not so closely engage the inner surface 30 of the rotating cutting blade as to become dislodged from the tenaculum 8 and entrapped in the axial bore 24 of the cutting blade. Furthermore, it may be desired to use in the morcellator a stationary inner cylindrical sleeve 61, as shown in FIG. 12 of the drawings. The increased diameter of the adjoining portion 60 of the cutting blade is sufficient to receive the inner sleeve 61, also preferably having an inner diameter which is greater than the diameter of the sharpened edge 36 of the cutting blade 20 so that tissue morsels cut by blade 20 will not become dislodged in inner sleeve 61 and may pass freely through the axial bore 63 defined thereby.

As is evident from the foregoing description, the morcellator cutting blade 20 of the present invention minimizes metal-to-metal contact between the morcellator blade edge 36 and the tenaculum 8 to prevent the blade from dulling, flaring or chipping during the insertion and withdrawal of the tenaculum. The various blade designs described herein minimize the chance that the tenaculum 8 will damage the blade 20 during a laparoscopic surgical procedure. The enlarged diameter adjoining portion 60 of the cutting blade minimizes the chance that transected tissue morsels will become entrapped in the morcellator, and the two-section design of the cutting blade 20 reduces the overall production costs of the morcellator by allowing the more expensive materials to be used only where needed, such as where surgical stainless steel is used only at the distal end portion 44 of the cutting blade as opposed to over the full length of the morcellator cutting blade 20 as in conventional morcellators.

Although illustrative embodiments of the present invention have been described herein with reference to the accompanying drawings, it is to be understood that the invention is not limited to those precise embodiments, and that various other changes and modifications may be effected therein by one skilled in the art without departing from the scope or spirit of the invention.

While the foregoing is directed to embodiments of the present invention, other and further embodiments of the invention may be devised without departing from the basic scope thereof, which is only limited by the scope of the claims that follow. For example, the present invention contemplates that any of the features shown in any of the embodiments described herein, or incorporated by reference herein, may be incorporated with any of the features shown in any of the other embodiments described herein, or incorporated by reference herein, and still fall within the scope of the present invention.

What is claimed is:

1. A cutting blade for a surgical morcellator, the cutting blade having a distal end and a sharpened cutting edge situated at the distal end for transecting tissue to form tissue morsels, the cutting blade being structured to minimize contact between the sharpened edge and a tissue grasping instrument used with the morcellator during a surgical procedure, the cutting blade comprising:
a generally cylindrically-shaped sidewall defining an axial bore for the passage of transected tissue morsels therethrough, the sidewall having an outer surface residing generally in a first cylindrical plane, an inner surface residing generally in a second cylindrical plane, and
a sloped surface, the inner surface of the sidewall being disposed radially inwardly of the outer surface of the sidewall, the sloped surface extending transversely between the inner surface and the outer surface in the direction of the distal end of the cutting blade and joining the outer surface at an acute angle to define with the outer surface the sharpened cutting edge, the sharpened cutting edge residing in the first cylindrical plane in which the outer surface of the sidewall generally resides, wherein the sloped surface of the cutting blade sidewall extends radially inwardly beyond the second cylindrical plane in which the inner surface of the cutting blade sidewall generally resides to define a circular protrusion situated radially inwardly of the inner surface for selective engagement with the tissue grasping instrument used with the morcellator during a surgical procedure.

2. A cutting blade for a surgical morcellator as defined by claim 1, wherein the sloped surface of the cutting blade forms with the second cylindrical plane in which the inner surface of the cutting blade sidewall generally resides an exterior angle of about 15 degrees (15.degree.).

3. A cutting blade for a morcellator as defined by claim 1, wherein the cutting blade and the sloped surface defining the sharpened cutting edge are rotatable.
